(19) United States
(12) Patent Application Publication
(10) Pub. No.: US 2012/0283548 A1
(43) Pub. Date: Nov. 8, 2012

(54) METHOD AND DEVICE TO ASSIST A PATIENT IN RE-ASSUMING A DEFINED BREATH HOLD POSITION FOR AN IMAGE ACQUISITION

(76) Inventor: Andreas Greiser, Erlangen (DE)

(21) Appl. No.: 13/464,136

(22) Filed: May 4, 2012

(30) Foreign Application Priority Data
May 5, 2011 (DE) 10 2011 075 289.7

(51) Int. Cl. A61B 5/055; A61B 5/113

(52) U.S. Cl. 600/411

(57) ABSTRACT

In a method and device to assist a patient in the resumption of a defined breath hold position for an image acquisition, in particular a magnetic resonance image acquisition, reference data describing the defined breath hold position are acquired in a first breath hold phase by a measurement device, measurement data describing the current breath hold position are acquired with the measurement device and are compared with the reference data in the preparation of an additional breath hold phase, and information describing the deviation of the measurement data from the reference data is emitted by an output device in a humanly-perceptible form.

### BACKGROUND OF THE INVENTION

1. Field of the Invention

The present invention concerns a method and a device to assist a patient to re-assume a defined breath hold position for an image acquisition, in particular a magnetic resonance image acquisition, as well as a magnetic resonance installation with such a device.

2. Description of the Prior Art

In magnetic resonance imaging, particularly imaging of the heart (cardio imaging), it is known to use breathing commands in order to avoid image artifacts due to the breathing motion. The patient to be examined ideally holds his or her breath for the entire duration of the data acquisition. However, due to the limited duration of such a breath hold phase, it is often not possible to acquire all necessary image data in such a breath hold phase, which image data are to be later assembled in a reconstruction and/or an evaluation. Since the breath hold position is not reproducible without additional measures, registration problems arise in the assembly of the data, or the data are inconsistent.

To solve this problem, it has previously been proposed to use registration algorithms in order to retroactively adapt the image data respectively acquired during different breath hold phases to one another. This is complicated and not always fruitful, particularly when the breath hold positions deviate from one another.

### SUMMARY OF THE INVENTION

An object of the invention is to provide a method and a device that allow an optimally precise resumption of a previously-assumed breath hold position.

This object is achieved according to the invention by a method is provided to assist a patient in the resumption of a defined breath hold position for an image acquisition, in which reference data describing the defined breath hold position are acquired in a first breath hold phase by means of a measurement device, measurement data describing the current breath hold position are acquired with the measurement device and compared with the reference data in the preparation of an additional breath hold phase, and information describing the deviation of the measurement data from the reference data is provided to an output device that makes the information available in a humanly-perceptible form.

Therefore, the method according to the invention now enables an optimally precise reproduction of a desired reference breath hold position. For this purpose, a breath hold position that is used as a reference breath hold position is initially measured in a first measurement during a first breath hold phase. This breath hold position is described by reference data from which a reference value can be derived, for example. In the following breath hold phases in which additional data are to be acquired with the image acquisition device (in particular a magnetic resonance installation), a new measurement is implemented after the breathing command but before the image acquisition, and an instruction is provided to the patient via the output device as to how close the patient is to the reference breath hold position, such that the patient can orient himself or herself, so it consequently becomes easier for the patient to reassume the reference breath hold position described by the reference data. The method steps are implemented automatically, such that a cyclical, frequent updating and re-output of the information is possible that returns the patient to the reference breath hold position again in a simple manner.

The method provides the advantage that image data that are acquired in the different breath hold phases can be used particularly simply together within the scope of a reconstruction and/or evaluation because no (or only extremely slight) deviations occur. For example, image artifacts are thus avoided and fewer evaluation errors occur. Registration algorithms can be better used due to the significant contiguity of the data of the various breath hold phases.

According to the invention, multiple variants of measurement devices are conceivable that naturally can also be used cumulatively.

In a particularly advantageous embodiment of the present invention, a magnetic resonance installation is used as the measurement device with a navigator in a region comprising the respiratory organs (in particular the diaphragm) being acquired to determine at least a portion of the reference data and the measurement data. In this case, the magnetic resonance installation (that is also used for magnetic resonance image acquisition) is thus also the measurement device because the acquisition of the reference data, and the measurement data, takes place anyway before the actual (diagnostic) image acquisition. The use of a fundamentally known navigator as a measurement data acquisition thus is suitable. A navigator is a one-dimensional magnetic resonance acquisition that can be generated extremely quickly and with little effort. For example, if the diaphragm is acquired by means of the navigator, the movement of the diaphragm, and thus the breathing position, can be tracked by tracking an edge that marks the border of the diaphragm. It is particularly advantageous for a navigator acquired in the longitudinal direction to be used in order to be able to track an unambiguous edge of the diaphragm as a “marked” point. The position of such a marked point can also be used as a reference value or measurement value derived from the reference data or measurement data. This simplifies the comparison.

A breathing sensor can be used alternatively or additionally as a measurement device. In this case, the image acquisition device (in particular the magnetic resonance installation) itself is thus not used as a measurement device; rather, an external apparatus, namely the breathing sensor, is provided. Such breathing sensors are basically known. For example, within the scope of magnetic resonance compressed air cushions can be arranged, for example, between a local coil and the torso of the patient, so that the cushion is compressed by the breathing of the patient and a pressure measurement can then be made. Naturally, other types and variants of a breathing sensor are also conceivable and usable.

As noted, a reference value is determined from the reference data, and this reference value is compared with a measurement value determined from the measurement data. One example of such a reference value or measurement value is the position of a marked point of the diaphragm in a navigator, but generally all variables are suitable that can optimally precisely describe a breathing phase in its progression. In the case of an external breathing sensor that operates with a compressed air cushion, the pressure or the volume of displaced air can be used, for example.

It is particularly advantageous for the acquisition of the measurement data, the comparison and the output to be repeated continuously and/or cyclically in the additional breath hold phase until the defined breath hold position is resumed, possibly within a tolerance range. As already described, by such continuous assistance the patient is able to return again to the defined breath hold position (reference breath hold position), and a certain tolerance range can be defined within which the breath hold positions should be considered to be identical. The measurement rate is selected so that the time intervals are markedly shorter than the relevant time constants (for example 3-5 s), in particular are smaller than 100 ms. Given the use of navigators, the measurements can be made every 40 to 60 ms, but even shorter time intervals in which new measurement data are acquired are conceivable, for example 20 ms. A continuous measurement can also be provided in the case of a breathing sensor.

It must still be made known to the imaging system when the comparison indicates that the patient has re-assumed the defined breath hold position.

For this purpose, it is possible, upon the patient resuming the defined breath hold position, for the patient to confirm the correct position (possibly also after multiple attempts), for example by pressing an additionally provided button and/or by a previously agreed-upon gesture, whereupon the operator starts the actual image acquisition (for example a measurement protocol). In a preferred embodiment of the present invention, however, the image acquisition is started automatically given the resumption of a defined breath hold position (in particular within a tolerance range), in particular after holding the breath hold position for a predetermined time period. The processor that implements the comparison can communicate with, or be part of, the imaging system control unit. For example, it can thus be checked automatically whether the defined breath hold position is held for a predetermined time period (2 s, for example), whereupon the image acquisition can be started in entirely automated manner. No complex communication with an operator or an additional activation of control elements such as a button is then necessary.

In another advantageous embodiment of the present invention, the information is emitted as an acoustic signal. Because it is fundamentally difficult to reproduce optical information within the patient receptacle so that it is clearly visible to a patient, an acoustic signal is suggested in order to return the patient to the defined breath hold position. A tone that is switched on and off with a frequency can advantageously be used as a signal, the frequency being dependent on the deviation of the measurement data from the reference data and being increased as the measurement data more closely approaches the reference data, until a continuous tone is achieved. An acoustic signal that, like a proximity sensor in a motor vehicle, increases the on/off frequency the closer that the measurement data approaches to the reference data is thus provided to the patient. When the defined breathing position is achieved, a continuous tone sounds so that the patient detects that he or she should hold this current breathing position. By using a known signal concept, namely the proximity warning in a motor vehicle, for example as a parking aid, the patient can intuitively understand the meaning of the acoustic signal and can thus resume the defined breath hold position in a simple manner. A continuous tone is then emitted given agreement of the measurement data and the reference data, in particular within a tolerance range.

In principle, various output forms can be used as part of the output device in order to emit the acoustic signal, for example dedicated speakers, and/or headphones that the patient wears anyway in order to be able to receive instructions from the operator. However, in an additional embodiment of the present invention the acoustic signal is emitted by gradient coils of a magnetic resonance installation. In this case, the image acquisition device itself (here the magnetic resonance installation) can thus be used to also serve as an output device. The gradient coils are basically used as speakers by being activated with gradient pulses such that the oscillations resulting at the casing of the magnetic resonance installation, functioning as an oscillating body, produces the tone. Because the measurement of the breath hold position takes place outside of the actual image acquisition, such a “multiple use” of the gradient coils is feasible and avoids the provision of an external output means of the output device. In particular, if the magnetic resonance installation serves both as an output device and as a measurement device, it is conceivable to function without any additional external means. In this way a magnetic resonance installation can itself provide the necessary functionalities without requiring additional devices.

The information may also be made available optically, with such an optical output being reasonable primarily with regard to an operator. Naturally, it is also conceivable to arrange an optical display means (in particular a monitor) so that a patient positioned within the patient receptacle can also perceive the information and react accordingly to this, but the use of an acoustic signal is preferred.

In a further embodiment of the method according to the invention, after the measurement ends measurement data are newly acquired with the measurement device in a breath hold phase and are compared with the reference data. In this way it is possible to check whether the breathing position at the end of the image acquisition still corresponds to the breath hold position at the beginning of the breath hold phase. For example, an additional navigator can be implemented and acquired after the image acquisition has ended. By means of a breathing sensor it is also possible to check whether the defined breath hold position is being held during the entire image acquisition. For example, if one or more deviations that exceed thresholds occur, the image acquisition can be repeated and/or the data are discarded.

In addition to the method, the invention also concerns a device to assist the patient in reassuming a defined breath hold position for an image acquisition, having a measurement device, an output device and a control device. The control device is designed to implement the method according to the invention, meaning that it is designed to control a measurement device to acquire reference data in a first breath hold phase (in particular immediately before the image acquisition) and also to control the measurement device in the preparation of an additional breath hold phase to acquire measurement data describing the current breathing position. Within the control device the reference data can be compared with the measurement data, wherein the control device is designed to output information describing the deviation of the measurement data from the reference data to an output device. All statements with regard to the method according to the invention it can be analogously transferred to the device according to the invention so that the indicated advantages can also be achieved with this.

The measurement device can be a breathing sensor and/or a magnetic resonance installation. As described above, navigators can be acquired by means of the magnetic resonance installation (which is also used for image acquisition), from which navigators current breathing positions can be deduced.

The output device may also be an acoustic output device, in particular the gradient coils of a magnetic resonance installation, and/or can be an optical output device. A tone that is variable in its on/off frequency can be generated with such an acoustic output device, which tone, similar to a proximity warning in a motor vehicle, increasingly approaches a continuous tone the closer that one approaches to the desired, defined breath hold position (reference breath hold position). In particular, the gradient coils of the magnetic resonance installation that is also used for image acquisition can thereby be used in order to produce the tone.

The device can also include an operating device, in particular a button, that serves to provide a confirmation signal upon the patient assuming the defined breath hold position. In this way an operator can thus be informed that the patient has been successful in assuming the defined breath hold position again, and the acquisition can be started. Naturally, the control device can alternatively be designed to automatically start the image acquisition upon the breath hold position being resumed, in particular within a tolerance range, in particular after the breath hold position has been held for a predetermined time period.

The invention also concerns a magnetic resonance installation with a device according to the invention. In an embodiment of such a magnetic resonance installation, this is fashioned as a measurement device to acquire a navigator in the region of the respiratory organs may also serve an acoustic output device by means of its gradient coils. If both embodiments are realized, no complicated external components are required because the magnetic resonance installation acts both as a measurement device and as an output device. The control device can be the control device of the magnetic resonance installation that is provided anyway.

Otherwise, the statements with regard to the method according to the invention and the device according to the invention analogously apply to the magnetic resonance installation.

### BRIEF DESCRIPTION OF THE DRAWINGS

FIG. 1 shows the chronological workflow in the first breath hold phase in the method according to the invention.

FIG. 2 shows the chronological workflow in additional breath hold phases within the scope of the method according to the invention.

FIG. 3 is a schematic illustration of a device according to the invention.

FIG. 4 shows a magnetic resonance installation according to the invention.

### DESCRIPTION OF THE PREFERRED EMBODIMENTS

An exemplary embodiment of the method according to the invention should now be presented in detail in the following. This is to assist a patient who has assumed a first, defined breath hold position in a first breath hold phase in being able to achieve this defined breath hold position again even during additional breath hold phases.

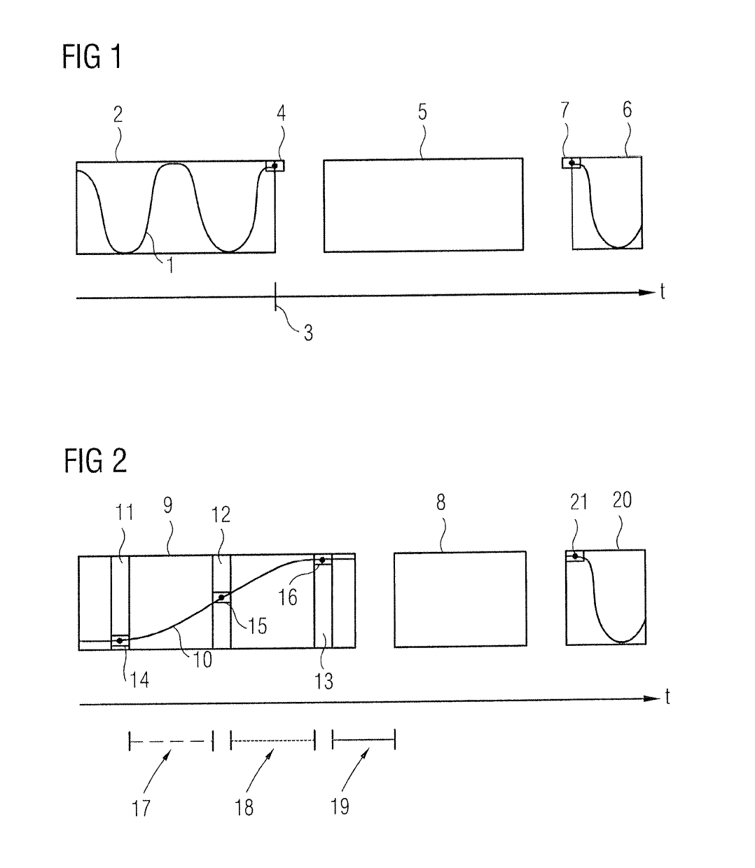

FIG. 1 shows a chronological workflow within the range of the first breath hold phase in the form of a principle drawing. In this exemplary embodiment, magnetic resonance data distributed across multiple breath hold phases should be acquired, which magnetic resonance data should then be assembled later for additional evaluation and/or reconstruction. A magnetic resonance installation that, in the present example, is also used as a measurement device within the scope of the method according to the invention is consequently used as an image acquisition device. A navigator is acquired in the longitudinal direction (thus head-to-foot direction) of the patient, which navigator shows a boundary of the diaphragm. This edge in the one-dimensional magnetic resonance image is the relevant variable that should be considered in the following.

The curve 1 in the Box 2 of FIG. 1 thus now shows precisely the curve of this edge in the navigator, which is also viewed later as a measurement value. A free breathing of the patient clearly takes place before this point in time, thus before the image acquisition and before a breath hold command. For the sake of a simpler presentation, the points in time of the acquisition of the individual navigators are not shown in the Box 2. The breath hold command to the patient, who holds his or her breath in a defined “navigator position” (which should be designated as a reference value 4 in the following) then takes place at a point in time 3. This reference breath hold position determined from the navigator acquired immediately before the (now following) image acquisition (Box 5) and to be considered as reference data consequently describes the defined breath hold position that should also be resumed during the following breath hold phases and corresponding image acquisitions.

In the exemplary embodiment, a navigator is also acquired again (Box 6) immediately after the image acquisition of magnetic resonance data (indicated by the Box 5), wherein a check can be made as to whether the navigator position—thus the measurement value 7 immediately after the image acquisition—still corresponds to the reference value 4, in particular within a tolerance range. If deviations that are too large occur, the acquired magnetic resonance data can be discarded and/or the image acquisition can be repeated.

After this first breath hold phase the reference value 4 based on the reference data is now consequently known. FIG. 2 now shows the curve for preparation and implementation of an additional breath hold phase that is used in turn for image acquisition according to Box 8. The preparation phase of this breath hold phase—and consequently of the image acquisition—is symbolized by Box 9, which is somewhat elongated for better presentation. The curve 10 within the Box 9 in turn reflects the measurement value in the navigator, meaning the position of an edge of the diaphragm as a marked point that is tracked in the one-dimensional magnetic resonance image that represents the navigator. For a better presentation, three acquisition points in time 11, 12 and 13 of navigators are also shown as examples, but it is noted that the navigators are acquired more frequently in the actual execution, for example at an interval of 100 ms or less.

The respective measurement values 14, 15 and 16 are now compared with the reference value 4 in a control device of the magnetic resonance installation. To assist the patient in thereby optimally resuming the defined breath hold position that corresponds to the reference value 4, an acoustic signal is output, for which a toner with a defined frequency is switched on and off. This frequency increases the closer that the measurement value 14, 15, 16 approaches the reference value 4. A continuous tone arises upon agreement of the measurement value 14, 15, 16 and the reference value 4, or if the measurement value lies within a tolerance range of one percent or less of the reference value 4, for example. This is likewise presented according to principle in FIG. 2. The measurement value 14 deviates strongly from the reference value 4, such that an acoustic signal 17 with a low frequency arises. The measurement value 15 already lies closer to the reference value 4, such that an acoustic signal 18 arises whose frequency is higher than that of the acoustic signal 15. The measurement value 16 coincides (possibly within the tolerance range) with the reference value 4, such that a continuous tone exists as an acoustic signal 19. Via the acoustic signals 17, 18 and 19, the patient thus receives information of how closely he has approached to the defined breath hold position and whether he has already achieved this. Given a continuous tone, the patient consequently no longer varies his or her breathing position and the additional breath hold phase begins.

If the control device of the magnetic resonance installation establishes that the breath hold position described by the reference value 4 has been held for a predetermined time period—two seconds, for example—the image acquisition begins in a wholly automated manner (Box 8).

In a further exemplary embodiment, however, it is also conceivable that the patient confirms the resumption of the breath hold position, for example by gesturing or by pressing an operating element (in particular a button) that was provided for this purpose. The operator can then start the image acquisition.

At this point it is noted further that it can by all means be possible that the patient requires multiple attempts to resume the defined breath hold position. This is possible without any problems because navigators are regularly (thus cyclically) acquired in the preparation phase (Box 9) in order to determine the deviation from the reference value 4 and two accordingly output the information, here as an acoustic signal. An additional optical output is also conceivable, for example a monitor for an operator.

Presently the acoustic signal 17, 18, 19 is moreover generated by the gradient coils of the magnetic resonance installation. The gradient coils are controlled with defined gradient pulses so that the casing (housing) of the magnetic resonance installation, for example, acts as a resonant body and generates the tone. Alternatively, it is naturally also conceivable—in particular when headphones are used anyway for the patient, for example—to use a different acoustic output means as part of the or as the output device, in particular the aforementioned headphones that are frequently provided anyway that the patient wears during the examination with the magnetic resonance installation.

After the image acquisition (Box 8), analogous to FIG. 1 a navigator is again acquired immediately after the image acquisition (Box 20) for a measurement value 21 in order to check whether the defined breath hold position was also maintained over the image acquisition.

The procedure that was described with regard to FIG. 2 can be repeated for an arbitrary number of additional breath hold phases.

It is noted again that, in addition to the navigators or as an alternative to these, a dedicated measurement device that is external to the magnetic resonance installation can also be used, for example a breathing sensor. Such a breathing sensor can comprise a compressed air cushion that is inserted between a local coil and the body of the patient and whose pressure or the volume of the displaced air is measured. Curves similar to the curves 1, 10 result.

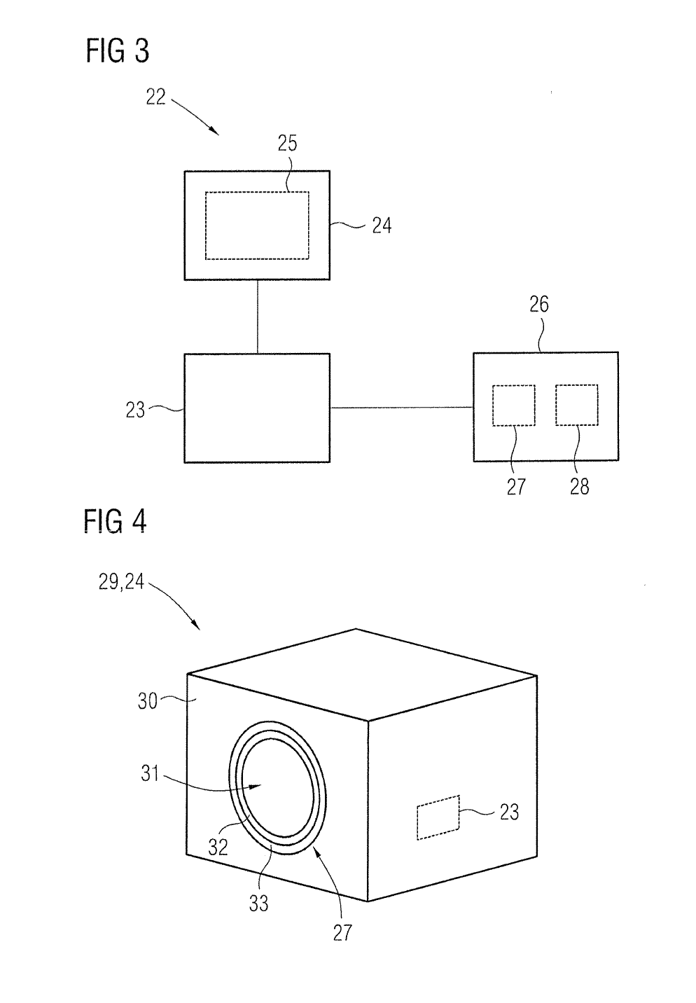

FIG. 3 now shows a principle drawing of a device according to the invention to assist the patient in resuming a defined breath hold position for an image acquisition, consequently a device with which the described method according to the invention can be executed.

Generally speaking, such a device 22 comprises a control device 23 that controls the operation of the entire device 22 and its components. The control device 23 can be the control device of the image acquisition device (in particular the magnetic resonance installation) that is used.

The assistance device 22 also has a measurement device 24 with which measurement data describing the current breathing position can be acquired, consequently the cited reference data and also the measurement data after the image acquisition and in additional breath hold phases. As already described, the measurement device 24 can be the magnetic resonance installation; however, it is also possible to use a breathing sensor 25 (indicated with dashed lines).

The output device 26 is additionally provided here with an acoustic output device 27 to emit the tone as an acoustic signal, and an optical output device 28. The acoustic output 27 can be formed, for example, by the gradient coils or by headphones to be worn by the patient, while the optical output device 28 can be a monitor of a display device of the image acquisition device (in particular the magnetic resonance installation); for example the device 22 can also have a control element (not shown in detail here)—in particular a button—for confirmation of the assumed, defined breath hold position by the patient.

It is relevant that the control device 23 is designed to implement the method according to the invention, meaning that it can control the measurement device 24 and the output device 26 so that the measurement data are acquired, which measurement data the control device 23 then compares, and the information is correspondingly output, in particular as an acoustic signal.

FIG. 4 shows a magnetic resonance installation 29 in which a device 22 according to the invention is integrated. As is typical, the magnetic resonance installation 29 has a basic field magnet unit 30 that has a patient receptacle 31 into which a patient can be moved for image acquisition. Surrounding the patient receptacle 31 are a radio-frequency coil 32 (body coil) and the gradient coils 33 that have multiple sub-coils for different directions. Here the gradient coils 33 act as an acoustic output device 27 while the magnetic resonance installation acts as a measurement device 24. Here the control device 23 serves both to control the typical function of the magnetic resonance installation 29 and the function of the device 22 integrated here in order to execute the method according to the invention.

Although modifications and changes may be suggested by those skilled in the art, it is the intention of the inventor to embody within the patent warranted hereon all changes and modifications as reasonably and properly come within the scope of his contribution to the art.

I claim as my invention:

1. A method to assist a patient in resumption of a defined breath hold position for an image acquisition, comprising:
in a first breath hold phase, using a measurement device to interact with a patient to acquire reference data that describe a defined breath hold position of the patient;
following said first breath hold phase, using the measurement device to acquire measurement data from the patient that describe a current breath hold position of the patient;
in a computerized processor, comparing said reference data with said measurement data to obtain a comparison result representing deviation of said measurement data from said reference data; and
from said comparison result, generating and emitting a humanly perceptible indication of said comparison result.

2. A method as claimed in claim 1 comprising using a magnetic resonance data acquisition device as said measurement device by acquiring a navigator in a region of the patient comprising respiratory organs, and determining at least a portion of at least one of said reference data and said measurement data from said navigator.

3. A method as claimed in claim 1 comprising using a breathing sensor that is configured to physically interact with the patient, as said measurement device.

4. A method as claimed in claim 1 comprising, in said processor, determining a reference value from said reference data and determining a measurement value from said measurement data, and implementing said comparison of said reference data with said measurement data by comparing said reference value with said measurement value.

5. A method as claimed in claim 1 comprising continuously or cyclically repeating acquisition of said measurement data and comparison of said measurement data with said reference data after said first breath hold phase until said defined breath hold position is resumed by the patient, within a predetermined tolerance range.

6. A method as claimed in claim 1 comprising also using said comparison result to automatically start acquisition of image data from the patient with an image data acquisition device, by starting said acquisition of image data when said comparison result indicates that the patient has resumed said defined breath hold position within a predetermined tolerance range.

7. A method as claimed in claim 6 comprising automatically starting said image acquisition when said patient has resumed said defined breath hold position within said predetermined tolerance range for a predetermined time.

8. A method as claimed in claim 1 comprising emitting an acoustic signal as said humanly perceptible indication of said comparison result.

9. A method as claimed in claim 8 comprising emitting said acoustical signal as an audible tone that is switched on and off with a frequency that increases as said measurement data more closely conform to said reference data, and emitting a continuous tone when said measurement data conform to said reference data within a predetermined tolerance range.

10. A method as claimed in claim 9 comprising using a magnetic resonance data acquisition device as said measurement device, and emitting said acoustic signal by operating gradient coils of said magnetic resonance data acquisition device dependent on said comparison result.

11. A method as claimed in claim 1 comprising acquiring image data from the patient when said comparison result indicates said reference data conform to said measurement data within a predetermined tolerance range and, after acquisition of said image data, again acquiring said measurement data and comparing said measurement data with said reference data to obtain a further comparison result, and evaluating said image data dependent on said further comparison result.

12. A device to assist a patient in resumption of a defined breath hold position for an image acquisition, comprising:
a measurement device configured to interact with a patient in a first breath hold phase to acquire reference data that describe a defined breath hold position of the patient;
following said first breath hold phase, the measurement device being configured to acquire measurement data from the patient that describe a current breath hold position of the patient;
a computerized processor configured to compare said reference data with said measurement data to obtain a comparison result representing deviation of said measurement data from said reference data; and
an output device configured to generate, from said comparison result, a humanly perceptible indication of said comparison result, and to emit said humanly perceptible indication of said comparison result.

13. A device as claimed in claim 12 wherein said measurement device is a breathing sensor configured to physically interact with the patient.

14. A device as claimed in claim 12 wherein said measurement device is a magnetic resonance data acquisition device.

15. A device as claimed in claim 14 wherein said magnetic resonance data acquisition device comprises gradient coils, and wherein said output device comprises said gradient coils and a control unit configured to operate the gradient coils to emit an acoustic signal as said humanly perceptible indication of said comparison result.

16. A device as claimed in claim 12 wherein said output device is an optical display configured to emit an optical presentation as said humanly perceptible indication of said comparison result.

17. A magnetic resonance apparatus to assist a patient in resumption of a defined breath hold position for an image acquisition, comprising:
a magnetic resonance data acquisition unit configured to receive a patient therein;
a control unit configured to operate said magnetic resonance data acquisition unit in a first breath hold phase to acquire reference data from the patient that describe a defined breath hold position of the patient;
said control unit being configured to operate said magnetic resonance data acquisition unit following said first breath hold phase to acquire measurement data from the patient that describe a current breath hold position of the patient;
a computerized processor configured to compare said reference data with said measurement data to obtain a comparison result representing deviation of said measurement data from said reference data;
an output device configured to generate, from said comparison result, a humanly perceptible indication of said comparison result, and to emit said humanly perceptible indication of said comparison result; and
said control unit being configured to receive a command when said comparison result indicates conformity between said measurement data and said reference data within a predetermined tolerance range, and to operate said magnetic resonance data acquisition unit upon receipt of said command to acquire magnetic resonance image data from the patient.

18. A magnetic resonance apparatus as claimed in claim 17 wherein said control unit is configured to operate said magnetic resonance data acquisition unit to acquire a navigator, as said measurement data, in a region of the patient comprising respiratory organs.

19. A magnetic resonance apparatus as claimed in claim 17 wherein said magnetic resonance data acquisition unit comprises gradient coils, and wherein said output device is a part of said control unit and is configured to operate said gradient coils to emit an acoustic signal as said humanly perceptible indication of said comparison result.

20. A magnetic resonance apparatus as claimed in claim 17 wherein said output device is a display device configured to display an optical presentation as said humanly perceptible indication of said comparison result.
